(19) United States
(12) Patent Application Publication
(10) Pub. No.: US 2026/0248753 A1
(43) Pub. Date: Aug. 27, 2026

(54) METHODS AND COMPOSITIONS COMPRISING CATIONIC LIPIDS FOR STIMULATING TYPE 1 INTERFERON GENES

(71) Applicant: PDS Biotechnology Corporation, Princeton, NJ (US)

(72) Inventors: Frank Bedu-Addo, Princeton, NJ (US); Gregory Conn, Princeton, NJ (US); Silva K. Gandhapudi, Princeton, NJ (US); Martin Ward, Princeton, NJ (US); Jerold Woodward, Princeton, NJ (US)

(21) Appl. No.: 19/539,744

(22) Filed: Feb. 13, 2026

Related U.S. Application Data
(63) Continuation of application No. 16/210,750, filed on Dec. 5, 2018, now Pat. No. 12,551,460.
(60) Provisional application No. 62/594,815, filed on Dec. 5, 2017.

(51) Int. Cl. A61K 31/21; A61K 39/12

(52) U.S. Cl. CPC A61K 31/21; A61K 39/12

(57) ABSTRACT

Methods and compositions for modifying type I IFN signaling pathways in a subject comprising the administration a cationic lipid to the subject are provided. The cationic lipids comprise 1,2-dioleoyl-3-trimethylammonium propane (DOTAP), N-1-(2,3-dioleoyloxy)-propyl-N, N,N-trimethyl ammonium chloride (DOTMA), 1,2-dioleoyl-sn-glycero-3-ethylphosphocholine (DOEPC), enantiomers and combinations thereof. The compositions may further comprise one or more antigenic components, wherein such components are autologous or nonautologous.

### CROSS-REFERENCE TO RELATED APPLICATIONS

This application is a continuation of U.S. patent application Ser. No. 16/210,750, filed on Dec. 5, 2018, which claims priority to and all the benefits of U.S. Provisional Patent Application No. 62/594,815, filed on Dec. 5, 2017, both of which are hereby expressly incorporated herein by reference in their entirety.

### BACKGROUND OF THE INVENTION

### 1. Field of the Invention

The present invention relates generally to methods and compositions for modifying type I IFN signaling pathways comprising the administration of cationic lipids.

### 2. Description of the Related Art

Type I interferons (IFN-I) in the last decade have been shown to be of critical importance in the mammalian immune response against disease. Type I interferons have been called “viral” interferons due to their direct induction by viral infections, in contrast to “immune” IFN, or IFN-γ, which is synthesized after receptor engagement of T cells and natural killer (NK) cells during immune responses. IFN-I are also well-documented inducers of tumor cell apoptosis and anti-angiogenesis via signaling through the interferon alpha receptor (IFNAR). Due to the critical role of IFN-I in the therapeutic immune response, significant emphasis is now focused on the identification of safe and effective methods of activating IFN-I in order to improve both preventive and therapeutic benefit of vaccines, immunotherapies and other immune based pharmaceuticals. For example, preclinical studies in mice have shown that IFNs-I directly activate other critical cells of the immune system, such as antigen-presenting dendritic cells (DC) and CD4 and CD8 T cells. IFNs-I also increase antigen presentation of the tumor cells to be recognized by T lymphocytes, hence presenting a key potential role in cancer immunotherapy.

In the field of vaccine development, methods and approaches that yield large numbers of potent antigen-specific cytotoxic T cells in humans in order to treat diseases caused by pathogens or to treat cancer, still remain an unmet medical need. Protein and peptide-based vaccines used in combination with immune stimulators such as adjuvants is one of the more promising approaches that have been evaluated to develop cytotoxic T cells. However, most adjuvants that are currently approved for human use are poor inducers of type I interferons and cytotoxic T cells, and have failed in establishing long lasting protective or therapeutic benefit.

Interferons (IFN-α/β/γ) are some of the more potent immune stimulatory cytokines, and play an important role in the development of CD8+ cytotoxic T cells. Interferons are also crucial for anti-viral immunity. Effective T cell immunity requires the activation of T cells with antigen (signal 1) in the presence of appropriate co-stimulatory signals (signal 2) and cytokines (signal 3) to drive a specific T cell immune response by promoting T cell expansion, survival, differentiation, and effector functions. IFN-I can act directly on T cells as signal 3 to induce T-cell activation and to drive their optimal expansion, survival and effector functions. Initially identified as anti-viral cytokines, IFN-I has been shown to enhance CD8 T cell expansion, survival and effector functions. Interferons also drive the maturation of antigen presenting cells by up regulating co-stimulatory molecules and by enhancing antigen cross presentation necessary for T cell activation. Furthermore, interferons have been demonstrated to have direct anti-tumor properties as well as indirect anti-tumor properties through IL-15 production. As a result of these significant immunological properties, various IFN-I inducing agents as well as recombinant IFN-I therapy are currently being tested in human trials.

Type I IFNs have been reported to prevent cellular transformation in vitro by sustaining expression of the tumor suppressor gene p53 (Takaoka A, Hayakawa S, Yanai H, Stoiber D, Negishi H, Kikuchi H, et al. Integration of interferon-α/β signaling to p53 responses in tumor suppression and antiviral defense. Nature (2003)424:516-23). Specific roles for IFN-I signaling in negatively regulating tumor cell proliferation and in triggering cell death in human cancer cell lines have also been demonstrated (Zitvogel L, Galluzzi L, Kepp O, Smyth M J, Kroemer G. Type I interferons in anticancer immunity. Nat Rev Immunol (2015) 15:405-14). In vivo, deletion of IFNAR1 from intestinal epithelial cells increased tumor formation in mice (Tschurtschenthaler M, Wang J, Fricke C, Fritz T M J, Niederreiter L, Adolph T E, et al. Type I interferon signaling in the intestinal epithelium affects Paneth cells, microbial ecology and epithelial regeneration. Gut (2014) 63:1921-31). Multiple studies have generated strong evidence that IFN-I induces antitumor effects predominantly via indirect stimulation of immune cells to rapidly eliminate malignant cells. Several studies strongly suggest that IFN-I promotes anticancer immune responses that are analogous to the reaction of the host against pathogens.

As a result of the ubiquitous IFNAR expression, IFN-I have been shown to have critical regulatory effects on immune cells in the context of inflammatory and viral diseases (Decker T, Müller M, Stockinger S. The yin and yang of type I interferon activity in bacterial infection. Nat Rev Immunol (2005) 5:675-87; Stetson D B, Medzhitov R. Type I interferons in host defense. Immunity (2006) 25:373-81). It is therefore evident that cellular mediators of the innate as well as the adaptive immune response may very well be regulated by IFN-I in protecting against malignant diseases.

IFN-I has been demonstrated to be important in tumor immune surveillance (Dunn G P, Bruce A T, Sheehan K C F, Shankaran V, Uppaluri R, Bui J D, et al. A critical function for type I interferons in cancer immunoediting. Nat Immunol (2005)6:722-9). As opposed to IFN-γ, IFN-I was found in bone marrow transfer experiments to act on host hematopoietic cells rather than the tumor cell itself during the induction of a protective antitumor immune response. Much has been reported on the mechanisms by which type I IFNs impact the cells of the innate and adaptive immune system in the context of tumor surveillance [Zitvogel L, Galluzzi L, Kepp O, Smyth M J, Kroemer G. Type I interferons in anticancer immunity. Nat Rev Immunol (2015) 15:405-14; Parker B S, Rautela J, Hertzog P J. Antitumour actions of interferons: implications for cancer therapy. Nat Rev Cancer (2016) 16:131-44)]. Several studies have identified a key role for type I IFNs, in the activation of host antigen presenting cells (Fuertes M B, Kacha A K, Kline J, Woo S-R, Kranz D M, Murphy K M, et al. Host type I IFN signals are required for antitumor CD8⁺ T cell responses through CD8α⁺ dendritic cells. J Exp Med (2011) 208:2005-16; Diamond M S, Kinder M, Matsushita H, Mashayekhi M, Dunn G P, Archambault J M, et al. Type I interferon is selectively required by dendritic cells for immune rejection of tumors. J Exp Med (2011) 208:1989-2003). It has been determined that the early produced type I IFNs act on the level of CD8α⁺ dendritic cells (DCs) that are required for the successful activation of tumor antigen-specific cytotoxic CD8+ T lymphocytes (CTLs). Type I IFN signaling specifically promotes the ability of CD8α⁺ DCs to cross-present antigens (Diamond M S, Kinder M, Matsushita H, Mashayekhi M, Dunn G P, Archambault J M, et al. Type I interferon is selectively required by dendritic cells for immune rejection of tumors. J Exp Med (2011) 208:1989-2003). It has been postulated that this effect is a result of the IFN-I to enhance the survival of DCs and therefore also enhance antigen persistence on the cell surface during cross-presentation (Lorenzi S, Mattei F, Sistigu A, Bracci L, Spadaro F, Sanchez M, et al. Type I IFNs control antigen retention and survival of CD8α(+) dendritic cells after uptake of tumor apoptotic cells leading to cross-priming. J Immunol (2011) 186:5142-50; Schiavoni G, Mattei F, Gabriele L. Type I interferons as stimulators of DC-mediated cross-priming: impact on anti-tumor response. Front Immunol (2013) 4:483). Type I IFNs promote DC maturation, differentiation, and migration (Fuertes M B, Woo S-R, Burnett B, Fu Y-X, Gajewski T F. Type I interferon response and innate immune sensing of cancer. Trends Immunol (2013) 34:67-73).

It is important to note that type I IFNs are able to induce the release of interleukin 15 (IL15) by DCs (Mattei F, Schiavoni G, Belardelli F, Tough DF. IL-15 is expressed by dendritic cells in response to type I IFN, double-stranded RNA, or lipopolysaccharide and promotes dendritic cell activation. J Immunol (2001) 167:1179-87). This promotes the survival of CD8⁺ memory cells and NK cells (Huntington N D. The unconventional expression of IL-15 and its role in NK cell homeostasis. Immunol Cell Biol (2014) 92:210-3). This effect is not restricted to NK cells. CTLs have also been shown to acquire full effector functions in response to type I IFNs (Curtsinger J M, Mescher M F. Inflammatory cytokines as a third signal for T cell activation. Curr Opin Immunol (2010) 22:333-40; Fuertes M B, Kacha A K, Kline J, Woo S-R, Kranz D M, Murphy K M, et al. Host type I IFN signals are required for antitumor CD8⁺ T cell responses through CD8α⁺ dendritic cells. J Exp Med (2011) 208:2005-16). Also by having the ability to impact other innate immune cell subsets such as neutrophils (Wu C-F, Andzinski L, Kasnitz N, Kröger A, Klawonn F, Lienenklaus S, et al. The lack of type I interferon induces neutrophil-mediated pre-metastatic niche formation in the mouse lung. Int J Cancer (2015) 137:837-47; Jablonska J, Wu C-F, Andzinski L, Leschner S, Weiss S. CXCR2-mediated tumor-associated neutrophil recruitment is regulated by IFN-β. Int J Cancer (2014) 134:1346-58), NKT, and γδ T cells (Woo S-R, Corrales L, Gajewski T F. Innate immune recognition of cancer. Annu Rev Immunol (2015) 33:445-74), type I IFNs exhibit tumor-growth limiting properties.

Type I IFNs are released very early during infections (Biron C A. Initial and innate responses to viral infections—pattern setting in immunity or disease. Curr Opin Microbiol (1999) 2:374-81), and are important regulators of innate immune cell subsets such as DCs and NK cells in anticancer host responses. For NK cells, type I IFNs have been demonstrated in viral infection to be important in the ability to generate early responses to the infection, and are thought to enhance NK cell cytotoxicity and cytokine production (Lee C K, Rao D T, Gertner R, Gimeno R, Frey A B, Levy D E. Distinct requirements for IFNs and STATI in NK cell function. J Immunol (2000) 165:3571-7; Nguyen K B, Salazar-Mather T P, Dalod M Y, Van Deusen J B, Wei X, Liew F Y, et al. Coordinated and distinct roles for IFN-alpha beta, IL-12, and IL-15 regulation of NK cell responses to viral infection. J Immunol (2002) 169:4279-87).

In humans, it is reported that type I interferons are usually triggered via the engagement of pathogen associated pattern recognition receptors such as toll like receptors, NOD like receptors, and Retinoic Acid Inducible Gene I like (RIG-I) receptors. In addition, a variety of cytosolic secondary messengers (c-GMP), and cytosolic nucleic acid sensors can also trigger the type I interferon production through the activation of Stimulator of Interferon Genes (STING) pathway.

It is thought that IFN-I may mediate anti-tumor activity in a variety of ways. For example, Interferon can directly act on tumor cells altering their ability to survive and grow [Parker, B. S., J. Rautela, and P. J. Hertzog, Antitumour actions of interferons: implications for cancer therapy. Nat Rev Cancer, 2016. 16(3): p. 131-443].

A number of methods are being investigated in humans to harness type I mediated immunological properties. The administration of recombinant IFN-I monotherapy has been clinically proven in the treatment of chronic hepatitis C by administration of recombinant interferon-alpha (Adrian M. Di Bisceglie, M. D., Paul Martin, M. D., Chris Kassianides, M. D., Mauricio Lisker-Melman, M. D., Linda Murray, R. N., Jeanne Waggoner, B. A., Zachary Goodman, M. D., Steven M. Banks, Ph.D., and Jay H. Hoofnagle, M. D., Recombinant Interferon Alfa Therapy for Chronic Hepatitis C, Nov. 30, 1989 N Engl J Med 1989; 321:1506-1510). Another approach involves the administration of toll-like-receptor (TLR) agonists such as double stranded RNAs (Poly I:C), CpG oligonucleotides, Imiquimod, single standard RNA, etc. to trigger endogenous production of type I interferons (Munir Akkaya, Billur Akkaya, Patrick Sheehan, Pietro Miozzo, Mukul Rawat, Mirna Pena, Ann S Kim, Olena Kamenyeva, Juraj Kabat, Chen-Feng Qi, Silvia Bolland, Akanksha Chaturvedi and Susan K Pierce, The Toll-like receptor ligand CpG-A induces type 1 interferons in B cells contrasting the proinflammatory inducing activity of CpG-B, J Immunol May 1, 2017, 198 (Supplement) 152.4). More recently, STING pathway agonists are gaining more importance as a focused type I interferon activator for anti-tumor immunity[Corrales, L., et al., The host STING pathway at the interface of cancer and immunity. J Clin Invest, 2016. 126(7): p. 2404-11].

There continues to remain however, a clear need for effective methods for promoting strong activation of the type I interferon pathway. Given the many therapeutic benefits associated with the consequences of activating the type I interferon pathway, improved methods for doing so are clearly desirable.

What is needed therefore, are improved methods and compositions for vaccines that enable the potent activation of IFN1 (IFN-α/β/γ). Furthermore, what is needed are improved methods and compositions such as vaccines that elicit a robust cytotoxic T cell immune response with or without an antigen, such as a peptide or protein antigen. Preferably, such compositions and vaccines incorporate the use of immunotherapeutic agents that are safe, easy to administer and have minimal side effects or toxicity.

### SUMMARY OF THE INVENTION

Provided herein are novel methods and compositions for vaccines comprising cationic lipids as potent activators of IFN1 (IFN-α/β/γ). As described herein, the present compositions enable the induction of an effective and robust cytotoxic T cell immune response when administered to a host. The compositions comprise cationic lipids, optionally combined with peptides or protein antigens; in certain embodiments, the cationic lipids are in the form of liposomes. As described in detail below, the compositions of the present invention result in IFN-I activated cytotoxic cells that are potent in regressing established tumors in mammals such as mice. The present invention further provides the use of cationic liposomes for use in specifically activating IFN-I in order to develop more effective preventions against infectious pathogens as well as therapies for cancer and other diseases.

Other features and advantages of the present invention will be readily appreciated, as the same becomes better understood, after reading the subsequent description taken in conjunction with the accompanying drawings.

### BRIEF DESCRIPTION OF THE DRAWINGS

FIG. 1 provides a graph demonstrating Versamune® (R-DOTAP) induced rapid and persistent recruitment of immune cells in the draining lymph nodes. The data in FIG. 1 corresponds to an experiment wherein C57BL/6J mice (n=4) were injected with 100 μl of 12 mM R-DOTAP liposome nanoparticles subcutaneously behind the neck. Mice injected with PBS, and LPS (50 μg/mouse) (24 h time point) were used as negative and positive controls respectively. 4 h or 24 hr after injection, axillary and brachial draining lymph nodes from each mouse were pooled and processed by collagenase digestion. CD11c positive cells (~20,000 cells) in these lymph node cell suspensions were sort-purified and lysed in RLT buffer and processed for relative gene expression analysis using nCounter® mouse inflammation kit and nanostring technologies. Data demonstrate up-regulation of the type I interferon genes by R-DOTAP and represent mean of 4 mice in each group.

FIG. 2 provides a graph demonstrating R-DOTAP induced type IFN gene expression in draining lymph nodes. The data in FIG. 2 corresponds to an experiment wherein C57BL/6J mice (n=2) were injected with 50 μl of 6 mM R-DOTAP or S-DOTAP nanoparticles subcutaneously in the foot pads. Mice injected with PBS, and LPS (50 μg/mouse) (24 h time point) were used as negative and positive controls respectively. 3 h or 24 hr after injection, popliteal draining lymph nodes from each mouse were pooled and lysed in RLT buffer and processed for relative gene expression analysis using Taqman gene expression assay and RT-PCR. Data demonstrate up-regulation of the type I interferon genes by both S-DOTAP and R-DOTAP.

FIGS. 3A, 3B, and 3C provide graphs demonstrating R-DOTAP induced cell recruitment and CD69 expression in draining lymph nodes. The data in FIG. 3 corresponds to an experiment wherein A) C57BL/6J mice (n=3) were injected with 50 μl of 6 mM R-DOTAP or 280 mM Sucrose subcutaneously in the foot pads. At indicated times popliteal draining lymph nodes from each mouse were isolated and single suspensions of lymph nodes were enumerated using hemocytometer to obtain total cell numbers. B) C57BL/6J (n=3) or IFNAR−/− (n=3) mice were injected with 50 μl of 6 mM R-DOTAP or 280 mM sucrose in the foot pad and draining popliteal lymph nodes were harvested from each mouse and enzymatically digested lymph nodes were assessed for the total cell number at 24 hr (B) using hemocytometer and expression of CD69 on CD3+ T cells (B) was assessed using flowcytometry. A-B) data represent total number of cells in each draining lymph node. C) Data represent percent CD 69 +cells each group 24 hr after vaccination (n=3 mice per group).
FIGS. 4A-4B provide graphs demonstrating the induction of type I interferon production by dendritic cells by cationic lipids. The data in FIG. 4A corresponds to an experiment wherein Bone marrow derived dendritic cells (BMDCs) from IFNAR-Ko mice were stimulated with indicated (6-400 μM) concentrations of cationic lipids or LPS positive control (1-500 ng/ml) for 24 hrs. The data in FIG. 4B corresponds to an experiment wherein FLT 3-induced BMDCs (pDCs) and GM-CSF/IL-4 derived BMDC (cBMDCs) were stimulated with indicated concentration (6-400 μM) concentrations of cationic lipid or LPS positive control (1-500 ng/ml) for 24 hrs. To measure type I interferon production, the cell supernatants (100 μl) were added to reporter cells (B16.Blue-IFNα/β cell from InvivoGen, USA) cultures and incubated for 18 hs to stimulate the type I interferon induced production of secreted alkaline phosphatase (SEAP) by reporter cells. The SEAP activity in the reporter cell supernatants was quantified using colorimetric SEAP assay kit according to the manufacture protocol. BMDC secreted type I Interferons were quantified using a standard curve is generated using recombinant mouse IFN-β stimulated SEAP activity in the reporter cell line.

FIGS. 5A-5B provide graphs demonstrating R-DOTAP based vaccine efficacy in type I IFN signal deficient mice. The data in FIG. 5A corresponds to an experiment wherein C57BL/6J mice (n=6) or IFNAR −mice from Jackson were injected subcutaneously with 100 μl of vaccine formulations (Versamune® or complete Freund's adjuvant) containing SIINFEKL peptide (SEQ ID NO: 1) or RF9 peptide. The data in FIG. 5B corresponds to an experiment wherein C57BL/6J mice (n=6) or IFNAR−/− mice from Jackson were injected subcutaneously with 100 μl of vaccine formulations (Versamune® or complete Freund's adjuvant) containing SIINFEKL peptide (SEQ ID NO: 2) on day 0 and day 7. Seven days after the second vaccination, spleen from the vaccinated and control mice was processed to measure antigen-specific CD8 T cell responses induced by the vaccine formulations in the wild type and IFNAR−/− mice. Antigen-specific CD8T cell responses were assessed by measuring SIINFEKL specific (FIG. 5A) (SEQ ID NO: 1) and RF9 peptide specific (FIG. 5BB) (SEQ ID NO: 2) CD8 T cell production of IFN-γ in an ELISPOT assay. Data is a representative of multiple experiments with similar results. Statistical significance of the data was estimated using one-way ANOVA, and multiple comparisons were analyzed using Tukey's testing. **Statistically significant (p<0.05) compared WT Versamune® vaccinated group.

### DETAILED DESCRIPTION

The present invention may be understood more readily by reference to the following detailed description of the specific embodiments included herein. Although the invention has been described with reference to specific details of certain embodiments thereof, it is not intended that such details should be regarded as limitations upon the scope of the invention.

The entire text of the references mentioned herein are hereby incorporated in their entireties by reference including U.S. Provisional Patent Application Ser. No. 62/594,815, U.S. Pat. Nos. 7,303,881, 8,877,206, 9,789,129, and U.S. patent application Ser. Nos. 14/344,327, 14/407,419, 14/429,123, and U.S. Pat. No. 15/775,680.

Provided herein are novel methods and compositions comprising the use of cationic lipids, for the modification of type I Interferon genes. In certain embodiments the type I Interferon genes are upregulated and induce or activate type I interferon signaling. In certain embodiments the type I Interferon genes are downregulated and depress or deactivate type I interferon signaling. Also provided are novel methods and compositions comprising the use of cationic lipids to promote and increase the potency of the disease-specific CD8+ T-cell population via the effect of the type I interferons. In certain embodiments, the methods and compositions claimed herein may comprise the use of disease-specific antigens intended to direct the cytolytic activity against specific infected or diseased cells.

Cationic liposomes have been extensively used in-vivo for delivering small molecular weight drugs, plasmid DNA, oligonucleotides, proteins, and peptides and also as vaccine adjuvants. In the present invention, as a result of studies performed to better understand how cationic lipids may interact with the immune system and the mechanistic reasons for the ability of certain cationic lipids to facilitate cross presentation, the unique ability of cationic lipids to upregulate type I interferons was discovered. It was reported that the early produced type I IFNs act on the level of CD8α⁺ dendritic cells (DCs) that are required for the successful activation of tumor antigen-specific cytotoxic CD8⁺ T lymphocytes (CTLs), and Type I IFN signaling specifically promotes the ability of CD8α⁺ DCs to cross-present antigens. This ability of cationic lipids to facilitate antigen cross presentation leading to antigen-specific CD8+ T-cell responses has now been demonstrated in both pre-clinical and human clinical studies. The ability to use cationic lipids to safely activate type I interferons to promote antigen cross presentation and induction of CD8+ T-cells in humans presents the potential to successfully addresses a significant unmet medical need in the field of therapeutic immunology.

In an embodiment contemplated herein, a cationic lipid is administered to activate the Type I interferon pathway in a subject.

In another embodiment in, a cationic lipid is combined with a disease-specific antigen to activate Type I interferons and to facilitate cross presentation of antigens to prime disease-specific CD8+ T-cells to treat a disease in a subject.

In another embodiment a method of treating cancer is provided wherein the method comprises the step of treating the subject with a Type I interferon-activating cationic lipid combined with a protein antigen.

In another embodiment a method of treating cancer is provided wherein the method comprises the step of treating the subject with a cationic lipid combined with a T-cell activating vaccine.

In another embodiment a method of treating cancer is provided wherein the method comprises the step of treating the subject with a cationic lipid combined with a protein or peptide tumor antigen, and in combination with an adjuvant.

In another embodiment a method of treating cancer is provided wherein the method comprises the step of treating the subject with a cationic lipid combined with any tumor antigen including a DNA or RNA-based antigen.

In another embodiment a method of treating cancer is provided wherein the method comprises the step of treating the subject with a cationic lipid to activate the type I interferon pathway optionally combined with a tumor antigen in combination with an adjuvant and/or any agent that combats tumor immune suppression via reduction of MDSC, Tregs or blocking of immune check points.

In another embodiment a method of treating cancer is provided wherein the method comprises the step of treating the subject with a cationic lipid-based vaccine combined with a DNA or RNA-based tumor antigen in combination with an adjuvant and/or any agent that combats tumor immune suppression.

In yet another embodiment, a method of augmenting an anti-tumor immune response in a mammal is provided. The method comprises the step of treating the mammal with a type I interferon-activating cationic lipid or with one or more cationic lipids together with growth factors in some cases such as GM-CSF and cytokines.

In the various embodiments, the composition comprises one or more lipids with at least one type I interferon-activating cationic lipid and at least one antigen. In certain embodiments, more than one antigen is included.

In an embodiment, methods and compositions for modifying a type I IFN signaling pathway in a subject, such as a mammal, are provided wherein the methods comprise administration of compositions comprising cationic lipids to the subject. In an embodiment, the cationic lipid comprises 1,2-dioleoyl-3-trimethylammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof. The compositions may comprise specific enantiomers of the cationic lipid. In an embodiment the cationic lipid is 1,2-dioleoyl-3-trimethylammonium propane (DOTAP), in an embodiment, the cationic lipid is the R enantiomer of DOTAP, (R)-1,2-dioleoyl-3-trimethylammonium propane (R-DOTAP). In certain embodiments, the compositions further comprise antigens, such as proteins or peptide antigens.

In certain embodiments, modification of the type I IFN signaling pathway in a subject comprises down upregulation or activation of the pathway. In certain embodiments, modification of the type I IFN signaling pathway in a subject comprises down regulation or deactivation of the pathway. In certain embodiments, the T-cell response is elevated, the antigen-specific CD8+ T cell responses are elevated, and/or the immunogenicity of the composition is enhanced. In an embodiment, a method for treating cancer in a subject is provided, wherein the method comprises the administration of a composition comprising a cationic lipid and wherein the administration of the cationic lipid results in the stimulation of a type I IFN signaling pathway.

### Antigens

In an embodiment, the novel methods claimed herein comprise the administration of cationic lipids with one or more autologous antigens such as antigens derived from a subject's own tumor. In another embodiment, the methods comprise the administration of composition comprising a cationic lipid in combination with one or more non-autologous antigen(s) such as, but not limited to, synthetic peptides, recombinant proteins, RNA or DNA or active fragments thereof. In each case the objective is to activate the type I interferons to facilitate induction of a strong antigen-specific CD8+ T-cell response. The antigen can be any disease-associated antigen known to one skilled in the art.

A “tumor-associated antigen,” as used herein is a molecule or compound (e.g., a protein, peptide, polypeptide, lipoprotein, lipopeptide, glycoprotein, glycopeptides, lipid, glycolipid, carbohydrate, RNA, and/or DNA, or active fragment thereof) associated with a tumor or cancer cell and which is capable of provoking an immune response (humoral and/or cellular) when expressed on the surface of an antigen presenting cell in the context of an MHC molecule. Tumor-associated antigens include self-antigens, as well as other antigens that may not be specifically associated with a cancer, but nonetheless enhance an immune response to and/or reduce the growth of a tumor or cancer cell when administered to an animal. Additional specific embodiments are provided herein.

A “microbial antigen,” as used herein, is an antigen of a microorganism and includes, but is not limited to, infectious virus, infectious bacteria, infectious parasites and infectious fungi. Microbial antigens may be intact microorganisms, and natural isolates, fragments, or derivatives thereof, synthetic compounds which are identical to or similar to naturally-occurring microbial antigens and, preferably, induce an immune response specific for the corresponding microorganism (from which the naturally-occurring microbial antigen originated). In a preferred embodiment, a compound is similar to a naturally-occurring microorganism antigen if it induces an immune response (humoral and/or cellular) similar to a naturally-occurring microorganism antigen. Compounds or antigens that are similar to a naturally-occurring microorganism antigen are well known to those of ordinary skill in the art such as, for example, a protein, peptide, polypeptide, lipoprotein, lipopeptide, glycoprotein, glycopeptides, lipid, glycolipid, carbohydrate, RNA, and/or DNA. Another non-limiting example of a compound that is similar to a naturally-occurring microorganism antigen is a peptide mimic of a polysaccharide antigen. More specific embodiments are provided herein.

The term “antigen” is further intended to encompass peptide or protein analogs of known or wild-type antigens such as those described in this specification. The analogs may be more soluble or more stable than wild type antigen, and may also contain mutations or modifications rendering the antigen more immunologically active. Antigen can be modified in any manner, such as adding lipid or sugar moieties, mutating peptide or protein amino acid sequences, mutating the DNA or RNA sequence, or any other modification known to one skilled in the art. Antigens can be modified using standard methods known by one skilled in the art.

Also useful in the compositions and methods of the present invention are peptides or proteins which have amino acid sequences homologous with a desired antigen's amino acid sequence, where the homologous antigen induces an immune response to the respective tumor, microorganism or infected cell.

In an embodiment, the methods described herein comprise compositions wherein the cationic lipid is administered without an antigen to activate the type I interferons in order to facilitate a desired immune response to treat a disease in a subject, for example by activating natural killer cells to attack and kill diseased or infected cells. In another embodiment, the methods of the present invention comprise compositions wherein the cationic lipid is administered with an antigen(s), wherein the antigen may be associated with a tumor or cancer, i.e., a tumor-associated antigen, to develop an immunotherapeutic to prevent or treat a tumor. As such, in one embodiment, the tumor or immunotherapy of the present invention further comprises at least one epitope of at least one tumor-associated antigen. In another embodiment, the immunotherapeutic methods of the present invention further comprise a plurality of epitopes from one or more tumor-associated antigens. The tumor-associated antigens used with the cationic lipids and methods of the present invention can be inherently immunogenic, or non-immunogenic, or slightly immunogenic. As demonstrated herein, even tumor-associated self-antigens may be advantageously employed in the subject immunotherapies for therapeutic effect, since the subject compositions are capable of activating the Type I interferons (IFNs) which are known to mediate antitumor effects against several tumor types. Exemplary antigens include, but are not limited to, synthetic, recombinant, foreign, or homologous antigens, and antigenic materials may include but are not limited to proteins, peptides, polypeptides, lipoproteins, lipopeptides, lipids, glycolipids, carbohydrates, RNA and DNA. Examples of such therapies include, but are not limited to the treatment or prevention of breast cancer, head and neck cancer, melanoma, cervical cancer, lung cancer, prostate cancer gut carcinoma, or any other cancer known in the art susceptible to immunotherapy. In such therapies it is also possible to combine the antigen with the cationic lipid without encapsulation.

Tumor-associated antigens suitable for use in the present invention include both naturally occurring and modified molecules which may be indicative of single tumor type, shared among several types of tumors, and/or exclusively expressed or overexpressed in tumor cells in comparison with normal cells. In addition to proteins, glycoproteins, lipoproteins, peptides, and lipopeptides, tumor-specific patterns of expression of carbohydrates, gangliosides, glycolipids, and mucins have also been documented. Exemplary tumor-associated antigens for use in cancer vaccines include protein products of oncogenes, tumor suppressor genes, and other genes with mutations or rearrangements unique to tumor cells, reactivated embryonic gene products, oncofetal antigens, tissue-specific (but not tumor-specific) differentiation antigens, growth factor receptors, cell surface carbohydrate residues, foreign viral proteins, and a number of other self-proteins.

Specific embodiments of tumor-associated antigens include, e.g., mutated or modified antigens such as the protein products of the Ras p21 protooncogenes, tumor suppressor p53 and HER-2/neu and BCR-abl oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO 1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. Also included as tumor-associated antigens herein are whole cell and tumor cell lysates as well as immunogenic portions thereof, as well as immunoglobulin idiotypes expressed on monoclonal proliferations of B lymphocytes for use against B cell lymphomas.

Tumor-associated antigens and their respective tumor cell targets include, e.g., cytokeratins, particularly cytokeratin 8, 18 and 19, as antigens for carcinoma. Epithelial membrane antigen (EMA), human embryonic antigen (HEA-125), human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. In cervical cancer, useful antigens could be encoded by human papilloma virus. Chromagranin-A and synaptophysin are antigens of neuroendocrine and neuroectodermal tumors. Of particular interest are aggressive tumors that form solid tumor masses having necrotic areas. The lysis of such necrotic cells is a rich source of antigens for antigen-presenting cells, and thus the subject therapy may find advantageous use in conjunction with conventional chemotherapy and/or radiation therapy.

Tumor-associated antigens can be prepared by methods well known in the art. For example, these antigens can be prepared from cancer cells either by preparing crude extracts of cancer cells (e.g., as described in Cohen et al., Cancer Res., 54:1055 (1994)), by partially purifying the antigens, by recombinant technology, or by de novo synthesis of known antigens. The antigen may also be in the form of a nucleic acid encoding an antigenic peptide in a form suitable for expression in a subject and presentation to the immune system of the immunized subject. Further, the antigen may be a complete antigen, or it may be a fragment of a complete antigen comprising at least one epitope.

Antigens derived from pathogens known to predispose to certain cancers may also be advantageously included in the cancer vaccines of the present invention. It is estimated that close to 16% of the worldwide incidence of cancer can be attributed to infectious pathogens; and a number of common malignancies are characterized by the expression of specific viral gene products. Thus, the inclusion of one or more antigens from pathogens implicated in causing cancer may help broaden the host immune response and enhance the prophylactic or therapeutic effect of the cancer vaccine. Pathogens of particular interest for use in the cancer vaccines provided herein include the, hepatitis B virus (hepatocellular carcinoma), hepatitis C virus (heptomas), Epstein Barr virus (EBV) (Burkitt lymphoma, nasopharynx cancer, PTLD in immunosuppressed individuals), HTLVL (adult T cell leukemia), oncogenic human papilloma viruses types 16, 18, 33, 45 (adult cervical cancer), and the bacterium Helicobacter pylori (B cell gastric lymphoma). Other medically relevant microorganisms that may serve as antigens in mammals and more particularly humans are described extensively in the literature, e.g., C. G. A Thomas, Medical Microbiology, Bailliere Tindall, Great Britain 1983, the entire contents of which is hereby incorporated by reference.

In another embodiment, the antigen comprises an antigen derived from or associated with a pathogen, i.e., a microbial antigen. As such, in one embodiment, the pathogen vaccines of the present invention further comprise at least one epitope of at least one microbial antigen. Pathogens that may be targeted by the subject immunotherapies include, but are not limited to, viruses, bacteria, parasites and fungi. In another embodiment, the pathogen vaccines of the present invention further comprise a plurality of epitopes from one or more microbial antigens.

The microbial antigens finding use in the cationic lipid immunotherapies and methods may be inherently immunogenic, or non-immunogenic, or slightly immunogenic. Exemplary antigens include, but are not limited to, synthetic, recombinant, foreign, or homologous antigens, and antigenic materials may include but are not limited to proteins, peptides, polypeptides, lipoproteins, lipopeptides, lipids, glycolipids, carbohydrates, RNA, and DNA.

Exemplary viral pathogens include, but are not limited to, viruses that infect mammals, and more particularly humans. Examples of virus include, but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Also, gram negative and gram positive bacteria may be targeted by the subject compositions and methods in vertebrate animals. Such gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borella burgdorferi, Legionella pneumophiliaii, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatumii, Streptobacillus moniliformis, Treponema pallidium, Treponema pertenue, Leptospira, Rickettsia, and Actinomyces israelli.

Polypeptides of bacterial pathogens which may find use as sources of microbial antigens in the subject compositions include but are not limited to an iron-regulated outer membrane protein, (“IROMP”), an outer membrane protein (“OMP”), and an A-protein of Aeromonis salmonicida which causes furunculosis, p57 protein of Renibacterium salmoninarum which causes bacterial kidney disease (“BKD”), major surface associated antigen (“msa”), a surface expressed cytotoxin (“mpr”), a surface expressed hemolysin (“ish”), and a flagellar antigen of Yersiniosis; an extracellular protein (“ECP”), an iron-regulated outer membrane protein (“IROMP”), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda;and surface antigen of Ichthyophthirius; and a structural and regulatory protein of Cytophaga columnari; and a structural and regulatory protein of Rickettsia.Such antigens can be isolated or prepared recombinantly or by any other means known in the art.

Examples of pathogens further include, but are not limited to, fungi that infect mammals, and more particularly humans. Examples of fungi include, but are not limited to: Cryptococcus neoformansi, Histoplasma capsulatum, Coccidioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e. protists) include Toxoplasma gondii. Polypeptides of a parasitic pathogen include but are not limited to the surface antigens of Ichthyophthirius.

Other medically relevant microorganisms that serve as antigens in mammals and more particularly humans are described extensively in the literature, e.g., see C. G. A Thomas, Medical Microbiology, Bailliere Tindall, Great Britain 1983, the entire contents of which is hereby incorporated by reference. In addition to the treatment of infectious human diseases and human pathogens, the compositions and methods of the present invention are useful for treating infections of nonhuman mammals. Many vaccines for the treatment of non-human mammals are disclosed in Bennett, K. Compendium of Veterinary Products, 3rd ed. North American Compendiums, Inc., 1995; see also WO 02/069369, the disclosure of which is expressly incorporated by reference herein.

Exemplary non-human pathogens include, but are not limited to, mouse mammary tumor virus (“MMTV”), Rous sarcoma virus (“RSV”), avian leukemia virus (“ALV”), avian myeloblastosis virus (“AMV”), murine leukemia virus (“MLV”), feline leukemia virus (“FeLV”), murine sarcoma virus (“MSV”), gibbon ape leukemia virus (“GALV”), spleen necrosis virus (“SNV”), reticuloendotheliosis virus (“RSV”), simian sarcoma virus (“SSV”), Mason-Pfizer monkey virus (“MPMV”), simian retrovirus type 1 (“SRV-1”), lentiviruses such as HIV-1, HIV-2, SIV, Visna virus, feline immunodeficiency virus (“FIV”), and equine infectious anemia virus (“EIAV”), T-cell leukemia viruses such as HTLV-1, HTLV-II, simian T-cell leukemia virus (“STLV”), and bovine leukemia virus (“BLV”), and foamy viruses such as human foamy virus (“HFV”), simian foamy virus (“SFV”) and bovine foamy virus (“BFV”).

In some embodiments, “treatment,” “treat,” and “treating,” as used herein with reference to infectious pathogens, refer to a prophylactic treatment which increases the resistance of a subject to infection with a pathogen or decreases the likelihood that the subject will become infected with the pathogen; and/or treatment after the subject has become infected in order to fight the infection, e.g., reduce or eliminate the infection or prevent it from becoming worse.

Microbial antigens can be prepared by methods well known in the art. For example, these antigens can be prepared directly from viral and bacterial cells either by preparing crude extracts, by partially purifying the antigens, or alternatively by recombinant technology or by de novo synthesis of known antigens. The antigen may also be in the form of a nucleic acid encoding an antigenic peptide in a form suitable for expression in a subject and presentation to the immune system of the immunized subject. Further, the antigen may be a complete antigen, or it may be a fragment of a complete antigen comprising at least one epitope.

In order to improve incorporation of the antigen into the cationic lipid vesicles and also to improve delivery to the cells of the immune system, the antigen may be modified to increase its hydrophobicity or the negative charge on the antigen. Hydrophobicity of an antigen may be increased such as, for example, by conjugating to a lipid chain or hydrophobic amino acids in order to improve it's the antigen's solubility in the hydrophobic acyl chains of the cationic lipid, while maintaining the antigenic properties of the molecule. The modified antigen can be a lipoprotein, a lipopeptide, a protein or peptide modified with an amino acid sequence having increased hydrophobicity, and combinations thereof. The modified antigen may have a linker conjugated between the lipid and the antigen such as, for example, an N-terminal .alpha. or .epsilon.-palmitoyl lysine may be connected to antigen via a dipeptide serine-serine linker. Further, the antigen may be manipulated to increase its negative charge by altering the formulation buffer in which the antigen is encapsulated into the cationic lipid complexes or by covalently attaching anionic moieties such as, for example, anionic amino acids to the antigen.

In some embodiments described herein, the cationic lipid may be in the form of nanoparticle assemblies. As used herein, the term “nanoparticle” refers to a particle having a size measured on the nanometer scale. As used herein, the “nanoparticle” refers to a particle having a structure with a size of less than about 10,000 nanometers. In some embodiments, the nanoparticle is a liposome.

The cationic lipid compositions of the present invention may form liposomes that are optionally mixed with antigen and may contain the chiral cationic lipids alone or chiral cationic lipids in combination with neutral lipids. Suitable chiral cationic lipid species include, but are not limited to the R and S enantiomers.

As used herein, the term “cationic lipid” refers to any of a number of lipid species which carry a net positive charge at physiological pH or have a protonatable group and are positively charged at pH lower than the pKa. Suitable cationic lipids according to the present disclosure may include, but are not limited to: 3-β[⁴N-(¹N,⁸-diguanidino spermidine)-carbamoyl]cholesterol (BGSC); 3-β[N,N-diguanidinoethyl-aminoethane)-carbamoyl]cholesterol; BGTC); N,N¹N₂N³Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N-dimethyl-1-p- -ropanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3-dioleoyloxy-1,4-butane-diammonium iodide) (Tfx-50); N-1-(2,3-dioleoyloxy) propyl-N,N,N-trimethyl ammonium chloride (DOTMA) or other N-(N,N-1-dialkoxy)-alkyl-N,N,N-trisubstituted ammonium surfactants; 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-.beta.-hydroxyethylammonium) or DORIE (DL-1,2-O-dioleoyl-3-dimethylaminopropyl-.beta.-hydroxyethylammoniu- -m) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES), cholesteryl-3.beta.-carboxyl-amido-ethylenetrimethylammonium iodide, 1-dimethylamino-3-trimethylammonio-DL-2-propyl-cholesteryl carboxylate iodide, cholesteryl-3-O-carboxyamidoethyleneamine, cholesteryl-3-.beta.-oxysuccinamido-ethylenetrimethylammonium iodide, 1-dimethylaminol-3-trimethylammonio-DL-2-propyl-cholesteryl-3-.beta.-oxysu-ccinate iodide, 2-(2-trimethylammonio)-ethylmethylamino ethyl-cholesteryl-3-.beta.-oxysuccinate iodide, 3-.beta.-N-(N′,N′-dimethylaminoethane) carbamoyl cholesterol (DC-chol), and 3-.beta.-N-(polyethyleneimine)-carbamoylcholesterol; O,O′-dimyristyl-N-lysyl aspartate (DMKE); O,O′-dimyristyl-N-lysyl-glutamate (DMKD); 1,2-dimyristyloxypropyl-3-dimethyl-hydroxy ethyl ammonium bromide (DMRIE); 1,2-dilauroyl-sn-glycero-3-ethylphosphocholine (DLEPC); 1,2-dimyristoyl-sn-glycero-3-ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-distearoyl-sn-glycero-3-ethylphosphocholine (DSEPC); 1,2-dioleoyl-3-trimethylammonium propane (DOTAP); dioleoyl dimethylaminopropane (DODAP); 1,2-palmitoyl-3-trimethylammonium propane (DPTAP); 1,2-distearoyl-3-trimethylammonium propane (DSTAP), 1,2-myristoyl-3-trimethylammonium propane (DMTAP); and sodium dodecyl sulfate (SDS). Furthermore, structural variants and derivatives of the any of the described cationic lipids are also contemplated.

In some embodiments, the cationic lipid is selected from the group consisting of DOTAP, DOTMA, DOEPC, and combinations thereof. In other embodiments, the cationic lipid is DOTAP. In yet other embodiments, the cationic lipid is DOTMA. In other embodiments, the cationic lipid is DOEPC. In some embodiments, the cationic lipid is purified.

In some embodiments, the cationic lipid is an enantiomer of a cationic lipid. The term “enantiomer” refers to a stereoisomer of a cationic lipid which is a non-superimposable mirror image of its counterpart stereoisomer, for example R and S enantiomers. In various examples, the enantiomer is R-DOTAP or S-DOTAP. In one example, the enantiomer is R-DOTAP. In another example, the enantiomer is S-DOTAP. In some embodiments, the enantiomer is purified. In various examples, the enantiomer is R-DOTMA or S-DOTMA. In one example, the enantiomer is R-DOTMA. In another example, the enantiomer is S-DOTMA. In some embodiments, the enantiomer is purified. In various examples, the enantiomer is R-DOEPC or S-DOEPC. In one example, the enantiomer is R-DOEPC. In another example, the enantiomer is S-DOEPC. In some embodiments, the enantiomer is purified.

It should be noted that for the purposes of illustration several examples are performed utilizing a model HPV antigen which has been well studied and which serves as a suitable antigen to illustrate the effect of type I interferon upregulation on antigen cross presentation to CD8+ T-cells.

In general, when referring to treatment, the compositions discussed herein may be administered orally, parenterally (e.g., intravenously or subcutaneous administration), by intramuscular injection, by intraperitoneal injection, transdermally, extracorporeally, by intracavity administration, transdermally, or topically or the like, including topical intranasal administration or administration by inhalant. The topical administration can be ophthalmically, vaginally, rectally, or intranasally. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

As used herein, “parenteral administration” of the composition, if used, is generally characterized by injection. Injectables can be prepared in conventional forms, either as liquid solutions or suspensions, solid forms suitable for solution of suspension in liquid prior to injection, or as emulsions. Parenteral administration includes use of a slow release, a time release or a sustained release system such that a constant dosage is maintained.

The term “therapeutically effective” means that the amount of the composition used is of sufficient quantity to ameliorate one or more causes or symptoms of a disease or disorder, such as aberrant cell growth, tumor development, and cancer. Such amelioration only requires a reduction or alteration, not necessarily elimination. Such amelioration may comprise the elicitation of an immune response. Effective dosages and schedules for administering the disclosed compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products.

The specific effective amount of the composition administered for any particular subject or patient will depend upon a variety of factors including the disease or disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts.

For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of ischemia-reperfusion injury, trauma, drug/toxicant induced injury, neurodegenerative disease, cancer, or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular subject or patient: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

The effective amount of a prescribed therapeutic may be given daily, every other day, weekly, monthly, bi-monthly, every other monthly, yearly, or at any other interval that is determined by the physician or provider to be effective. For example, the effective daily dose can be divided into multiple doses for purposes of administration. Consequently, single dose therapeutic can contain such amounts or submultiples thereof to make up the daily dose. Disclosed therapeutics can also be administered as part of a combination of anti-tumor or anti-cancer treatments. In an aspect, disclosed compositions can be administered to the subject or patient prior to treatment with an anti-tumor or anti-cancer treatment. In an aspect, disclosed compositions can be administered concurrently with the anti-tumor or anti-cancer treatment. In an aspect, disclosed composition can be administered subsequent to the anti-tumor or anti-cancer treatment. In an aspect, the patient or subject receives both treatments on an alternating or rotating schedule. In an aspect, the subject or patient receives a singular treatment with the disclosed composition. In an aspect, the subject or patient receives at least one treatment with the disclosed composition. In an aspect, the subject or patient receives at least one treatment with the disclosed composition and at least one other anti-tumor or anti-cancer treatment.

The dosage can be adjusted by the individual physician or the subject in the event of any counter-indications. Dosage can vary, and can be administered in one or more dose administrations daily, for one or several days. Guidance can be found in the literature for appropriate dosages for given classes of pharmaceutical products.

The terminology used herein is for the purpose of describing particular aspects only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, an aspect includes from the one particular value and/or to the other particular value. Similarly, when values are expressed as approximations, by use of the antecedent “about,” it will be understood that the particular value forms an aspect. It will be further understood that the endpoints of each of the ranges are significant both in relation to the other endpoint, and independently of the other endpoint.

The word “or” as used herein means any one member of a particular list and also includes any combination of members of that list.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75-100 percent as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100 percent, or more, or any amount of promotion in between compared to native or control levels. In an aspect, the increase or promotion is 10-20, 20-30, 30-40, 40-50, 50-60, 60-70, 70-80, 80-90, or 90-100 percent as compared to native or control levels. In an aspect, the increase or promotion is 0-25, 25-50, 50-75, or 75-100 percent, or more, such as 200, 300, 500, or 1000 percent more as compared to native or control levels. In an aspect, the increase or promotion can be greater than 100 percent as compared to native or control levels, such as 100, 150, 200, 250, 300, 350, 400, 450, 500 percent or more as compared to the native or control levels.

As used herein, the term “determining” can refer to measuring or ascertaining a quantity or an amount or a change in activity. For example, determining the amount of a disclosed polypeptide in a sample as used herein can refer to the steps that the skilled person would take to measure or ascertain some quantifiable value of the polypeptide in the sample. The art is familiar with the ways to measure an amount of the disclosed polypeptides and disclosed nucleotides in a sample.

The term “sample” can refer to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid). A sample may also be any body fluid or excretion (for example, but not limited to, blood, urine, stool, saliva, tears, bile) that contains cells or cell components.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing

from the scope and spirit of this invention. All changes, modifications, and variations coming within the meaning and range of equivalency of the claims are to be considered within their scope.

The present invention has been described in an illustrative manner. It is to be understood that the terminology, which has been used, is intended to be in the nature of words of description rather than of limitation.

Many modifications and variations of the present invention are possible in light of the above teachings. Therefore, within the scope of the appended claims, the present invention may be practiced other than as specifically described.

### EXAMPLES

For the Examples described below, the following materials, methods and protocols were utilized.

### Materials and Methods

Animals: Six to twelve-week-old C57BL6/J mice (B6 mice), B6. Cg-Tg(HLA-A/H2-D)2Enge/J transgenic breeder mice expressing human HLA-A2 gene (AAD mice), and IFNAR−/− (B6.129S2-Ifnar1tm1Agt/Mmjax) gene knockout breeder mice obtained from Jackson laboratories (Bar harbor, ME) were housed under specific-pathogen free conditions. Animal husbandry, breeding, and experimental procedures were performed as per IACUC protocols approved by the DLAR.

Peptides and Reagents: Peptides and Reagents: cGMP grade R-DOTAP and S-DOTAP (1, 2-dioleoyl-3-trimethyl ammonium-propane) were provided by Merck & Cie, Shaffhausen, Switzerland. cGMP grade R-DOTAP liposomal nanoparticles were produced by standard liposome manufacturing processes. Peptide antigens (KF18: KSSGQAEPDRAHYNIVTF (SEQ ID NO: 3), SF9: SIINFEKL (SEQ ID NO:1), RF9: RAHYNIVTF (SEQ ID NO:2) were synthesized and purified to >95% purity by GenScript, Piscataway, New Jersey, USA. All other cationic lipids were purchased from Avanti polar Lipids, Birmingham, AL.

Cell lines and bone marrow derived dendritic cells culture: Primary bone marrow derived dendritic cells were obtained by culturing the hematopoietic bone marrow cells for 8 days in complete RPMI media (RPMI media containing 10% FBS, 1 mM L-glutamine, 1 mM sodium pyruvate, 1X MEM non-essential amino acids, 50 μM-mercaptoethanol, 100 μ/ml penicillin, 100 μ/ml streptomycin) supplemented with recombinant mouse GM-CSF and IL-4 ( for conventional BMDCs (cDCs)) or recombinant flt3 ligand (for plasmacytoid BMDCs (pDCs)). B16-Blue™ IFN-α/β B Cells used for detecting cationic lipid induced type I IFN was purchased from InvivoGen, San Diego, California, USA, and cell cultures were maintained and according to the manufacturer's protocol.

IFN reporter cell assay and Cell phenotypic analysis: For detecting IFN, the supernatants from type I IFN producing cells were added to B16-Blue™ IFN-α/β Cells in a 96 well plate. After 24 hr incubation, the supernatants were assayed for SEAP activity using QUANTI-Blue™ reagent (InvivoGen, USA) according to manufacturer's instructions. Briefly, 50 μl of cell supernatants containing SEAP was mixed with 150 μl QUANTI-Blue™ reagent and incubated for 3-4 hr at 37° C. and absorbance was measured at 650 nm using a spectrometer. The concentrations of type I IFN in test samples were quantified using a standard curve generated from recombinant type I IFNβ treated B16-BlueTM IFN-α/β Cells in the same assay. To measure CD69 expression, single cell suspensions of lymph nodes were stained with fluorochrome conjugated CD3, and CD69 and percent CD69+ CD3+ T cells in each draining lymph node were measured using flowcytometry.

RNA isolation, Nano string Analysis, and gene expression assay: For gene expression analysis, draining popliteal lymph nodes (n=4) were enzymatically digested using cell dissociation cocktail for 60 min at 370C, and single cell suspensions were used to sort CD 11c+ ells using Sony SY3200 cell sorter. Sort purified CD11c+ cells (25K cells) were lysed in Qiagen's RLT cell lysis buffer (Qiagen, USA) and total RNA was isolated using Qiagen total RNA isolation kit (Qiagen, USA). Total RNA obtained was sent to University of Kentucky genomics core laboratory (Lexington, KY) where total RNA was mixed with ncounter TM mouse inflammation panel (Nano String Technologies, Seattle, WA) which can measure over 547 genes involved in immune response. Binding of mRNA was detected with Nanso String nCounter analysis system, and raw count data was normalized and statistically analyzed using SAS program by UK genomics core laboratory. For confirmation studies using RT-PCR, the lymph nodes were directly lysed in RLT cell lysis buffer, and the total isolation RNA was reverse transcribed to cDNA using QuantiTect reverses transcription kit (Qiagen, USA). The cDNA was then amplified using TaqMan gene expression system (Applied biosystems, USA) to detect and quantify mouse IFNα and IFNβ transcripts relative to GAPDH expression using quantitative PCR reaction.

Vaccination and Assessment of antigen specific T cell responses: Mice were anesthetized mice using isoflurane, and the injection site was shaved and cleaned with 70% ethanol prior to vaccination. For assessing antigen specific T cell response using ELISPOT, mice were vaccinated two doses (100 μl/dose) of vaccine formulation containing cationic lipid and antigenic peptides delivered at 7 day intervals or one dose of CFA formulation prepared by emulsifying equal volumes of complete freund's adjuvant and antigenic peptide delivered on day 0. Antigen specific responses were assessed 14 days after the first vaccine. For measuring antigen specific responses, spleen cells from the euthanized mice were used to detect antigen specific T cell responses using an IFN-γ ELISPOT Assay (Mabtech, Inc, Cincinnati, Ohio, USA). For IFN-γ ELISPOT assays, 2.5×10⁵ processed spleen cells were stimulated for 18-24 hr at 37° C. with CD8 T cell epitopes of interest in a 96 well plate pre-coated with the mouse IFN-γ capture antibody. After stimulation, wells were washed with PBS and incubated with biotin-conjugated anti-IFNγ antibody followed by the streptavidin-HRP antibody. To visualize the antigen specific IFN-y producing cells, wells were incubated for 6 minutes with TMB substrate, washed with water, and air dried. Spots were scanned and counted using CTL ImmunoSpot Analyzer and ImmunoSpot Ver. 4 software (Cellular Technology Limited, Cleveland, Ohio, USA). Spot counts were summarized as median values from triplicate samples. Each sample had unstimulated and a PMA/Ionomycin control wells to detect background and positive control. Wells were considered positive if the spot count exceeded 5 spots and antigen specific response as positive if the spot count is more than three-fold compared to controls.

### Example 1

### R-DOTAP Induces Type I Interferon Gene Expression in Draining Lymph Nodes

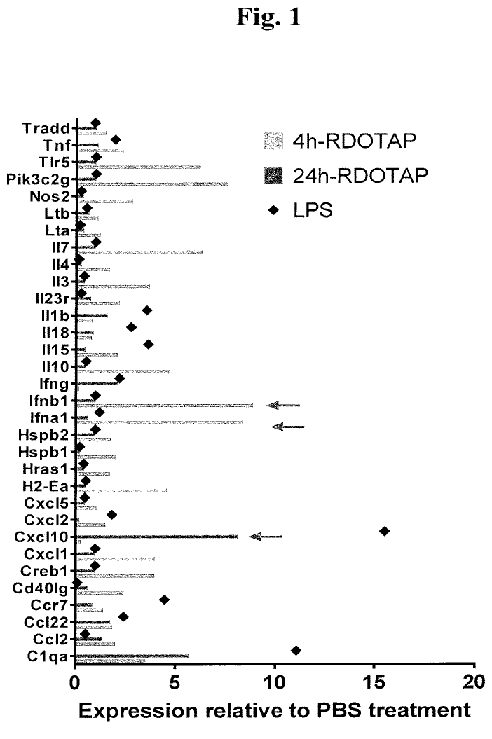

To assess cationic nanoparticle induced gene expression, C57BL/6J mice were injected with 100 μl of 12 mM R-DOTAP nanoparticles subcutaneously behind the neck, and inflammatory gene expression in draining lymph nodes analyzed 4 or 24 hours post vaccination. Mice injected with PBS, and LPS (50 μg/mouse) (24 h time point) were used as negative and positive controls respectively. 4 h or 24 hr after injection, axillary and brachial draining lymph nodes from each mouse were pooled and processed by collagenase digestion. Activated dendritic cells (CD11c⁺) cells in the lymph node cell suspensions were sort-purified and lysed in RLT buffer and processed for relative gene expression analysis using nCounter® mouse inflammation kit and nanostring technologies. R-DOTAP injection was found to significantly alter the expression of several inflammatory genes at both time points. Several genes including IFN-1α and IFN-1β showed increases greater than 5-fold higher than LPS which was used as a positive control. Expression of other genes including Cxcl10 were also significantly increased and more comparable to levels induced by LPS (FIG. 1).

### Example 2

### R-DOTAP and S-DOTAP Both Induce Interferon-a and Interferon-β Gene Expression in Draining Lymph Nodes

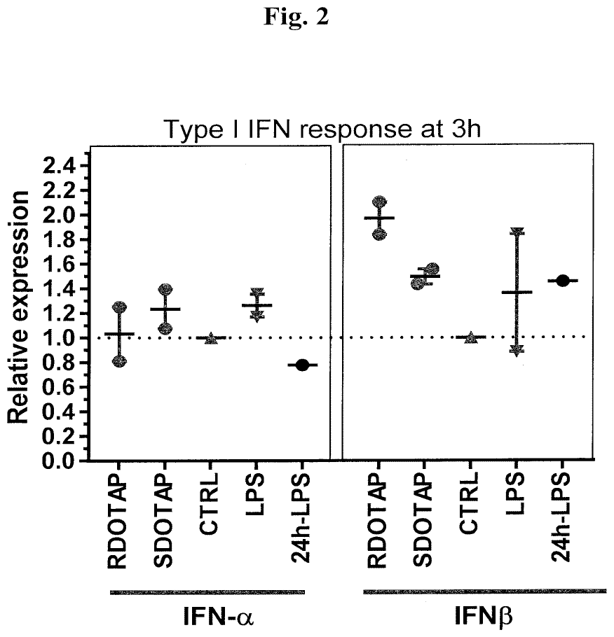

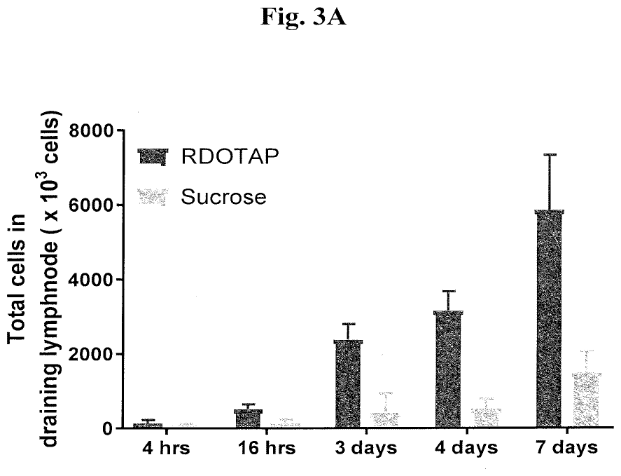

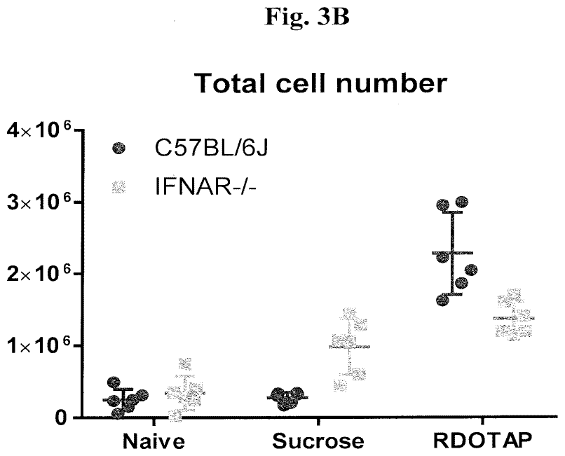

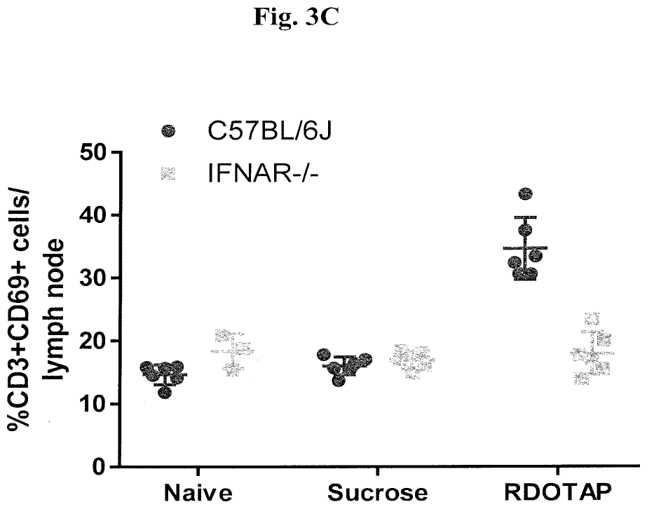

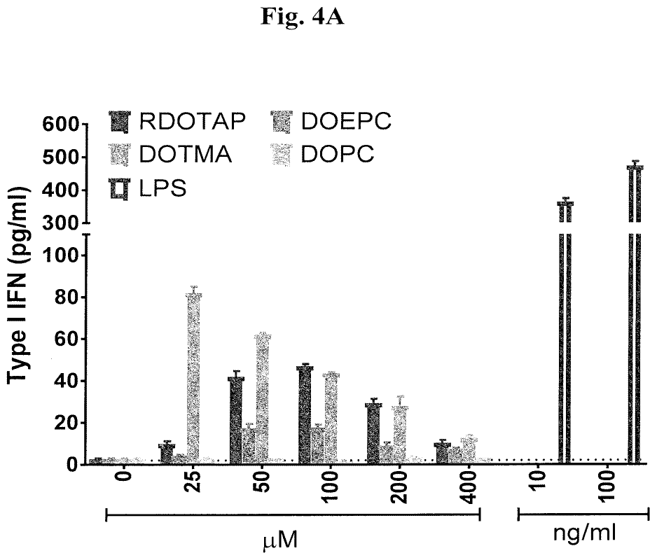

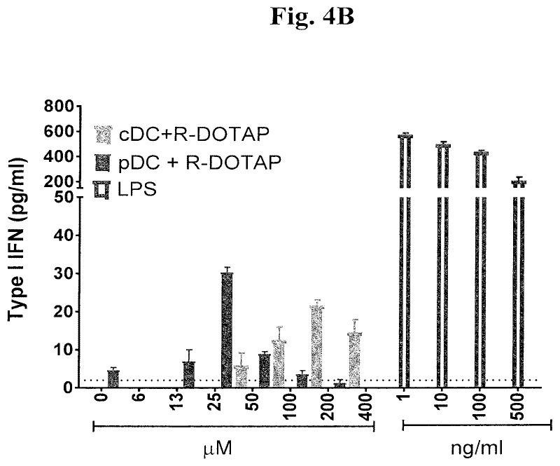

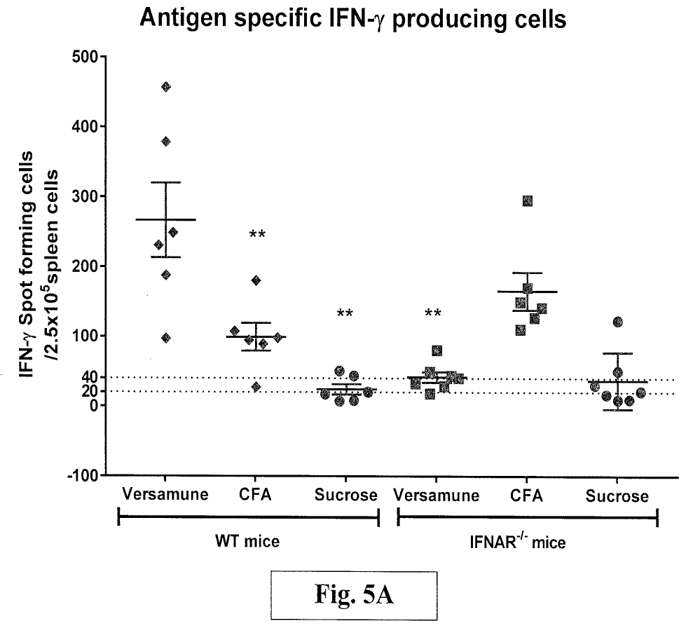

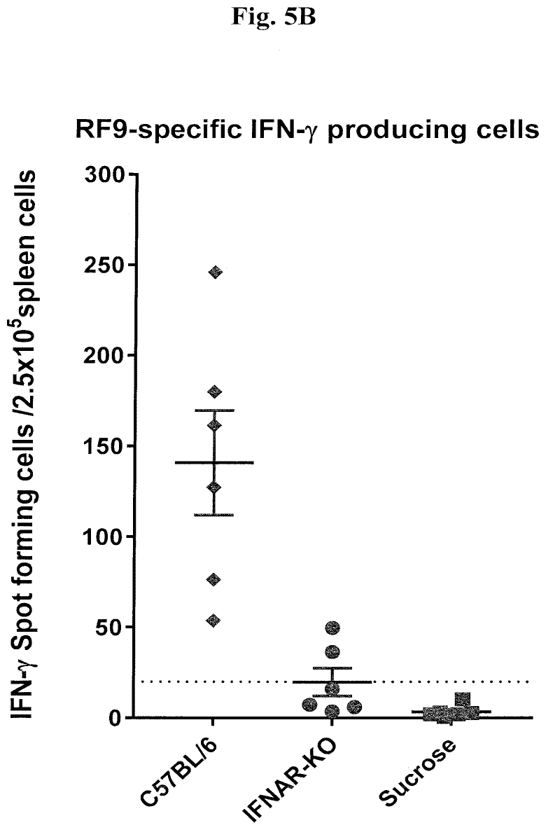

To compare type I Interferon gene expression by both R- and S-DOTAP, a similar study as outlined in Example 1 was performed. C57BL/6J mice were injected with 50 μl of 6 mM RDOTAP nanoparticles subcutaneously in the foot pads. Mice injected with PBS, and LPS (50 μg/mouse) (24 h time point) were used as negative and positive controls respectively. 3 h or 24 hr after injection, popliteal draining lymph nodes from each mouse were pooled and lysed in RLT buffer and processed for relative gene expression analysis using Taqman® gene expression assay and RT-PCR. The studies confirm that both R-and S-DOTAP are strong inducers of the type I interferons (FIG. 2).

### Example 3

### R-DOTAP and S-DOTAP Upregulate Type I Interferon-Associated Cd69 Expression on T-Cells

To further understand the effect and ability of cationic lipids to activate the type I interferons, C57BL/6J mice or IFNAR−/− mice were injected with 50 μl of 6 mM R-DOTAP nanoparticles or 6 mM S-DOTAP or 280 mM sucrose subcutaneously in the foot pads. 24 hr after injection, popliteal draining lymph nodes from each mouse were isolated and single cell suspensions of lymph nodes were stained with fluorochrome conjugated CD3 and CD69. R-DOTAP alone (without antigen) resulted in a visible increase in DLN size and this was due to a steady increase in total cell number over a 7-day period (FIG. 3A). Studies of T-Cell influx into the DLN with pertussis toxin suggest that lipids structurally related to R-DOTAP induce lymph node homing chemokines, most likely a direct result of type I IFN signaling. This increase in total cell number was confirmed to be dependent on type I IFN signaling as it was greatly reduced in the IFNaR knock-out mouse (FIG. 3B). Type I IFN is known to inhibit lymphocyte egress from lymphoid organs through the up-regulation of CD69 which, in turn, inhibits the sphingosine 1 phosphate receptor required for lymphocyte egress. We therefore postulated that injection of both S-DOTAP and R-DOTAP would result in the upregulation of CD69 in the DLN. Indeed, subcutaneous injection of both cationic lipids in wild type mice resulted in the up-regulation of CD69 most strongly in T cells. In contrast, no CD69 upregulation was seen after R-DOTAP injection of IFNaR knock-out mice, demonstrating that the R-DOTAP induced up regulation of CD69 is type I IFN dependent (FIG. 3C). The data represents % CD69+ CD3+ T cells in each draining lymph node.

### Example 4

### Various Cationic Lipids Upregulate Type I Interferons

Studies were performed in-vitro to evaluate the potential of various cationic lipids to induce the type I interferons. In this study the cationic lipids DOTAP, DOEPC and DOTMA were studied. The neutral lipid DOPC was also evaluated to confirm the specificity of the interferon up-regulation to cationic lipids. In one study, bone marrow derived dendritic cells (BMDCs) from IFNAR-Ko mice were stimulated with indicated (6-400 μM) concentrations of cationic lipids or LPS positive control (1-500 ng/ml) for 24 hrs (FIG. 4A). In a second study, FLT3-induced BMDCs (pDCs) and GM-CSF/IL-4 derived BMDC (cBMDCs) were stimulated with indicated concentration (6-400 μM) concentrations of R-DOTAP or LPS positive control (1-500 ng/ml) for 24 hrs (FIG. 4B). To measure type I interferon production, the cell supernatants (100 μl) were added to reporter cell (B16.Blue-IFNα/β B cell from invivogen, USA) cultures and incubated for 18hs to stimulate the type I interferon induced production of secreted alkaline phosphatase (SEAP) by reporter cells. The SEAP activity in the reporter cell supernatants was quantified using colorimetric SEAP assay kit according to the manufacture protocol. BMDC secreted type I Interferons were quantified using a standard curve is generated using recombinant mouse IFN-β stimulated SEAP activity in the reporter cell line. The studies confirm that the ability to upregulate the type I interferons is not specific to DOTAP but can be activated by multiple cationic lipids. It also confirms the inability of neutral lipids to activate the type I interferon pathway.

### Example 5

### Diminished Cationic Lipid Mediated Cd8+ T-Cell Response is Observed in Type I IFN-Deficient Mice

The demonstrated upregulation of Type I IFN-genes by cationic lipids, suggests that cationic lipids target the type I IFN pathway to induce robust CD8+ T-cell responses. To further confirm this hypothesis, an R-DOTAP formulation containing a well-characterized mouse CD8+ T-cell epitope (SIINFEKL (SEQ ID NO:1)) of chicken ovalbumin or HPV16-E7 protein derived CD8 T cell epitope (RF 9: RAHYNIVTF (SEQ ID NO:2)) was administered to wild-type C57BL 6J mice and to type I interferon signal deficient IFNAR^{-/-} mice on days 0 and on day 7. Mice were also vaccinated with complete Freund's adjuvant (CFA)+SIINFEKL. The peptide-only vaccine was used as a positive control. On day 14, antigen-specific (SIINFEKL) CD8+ T cell responses induced by R-DOTAP or CFA were assessed using the IFN-γ ELISPOT assay. As shown in FIG. 5A, vaccinating wild-type mice with the DOTAP formulation induced robust antigen-specific CD8+ T cell responses that are higher in magnitude than the CFA-adjuvanted formulation. In contrast, the CD8+ T cell response induced by R-DOTAP is dramatically reduced in the IFNAR−/− mice, although no such difference is noted with the CFA-adjuvanted formulation. We observed similar results (FIG. 5B) even with a tumor associated antigen expressed by HPV16 positive tumors. It is well documented that CFA adjuvant targets multiple TLR pathways and hence can bypass the requirement for type I IFN signaling to induce antigen-specific CD8+ T cell responses.

The loss of the cationic lipid-induced efficacy in the mice devoid of type I IFN signaling, coupled with the demonstrated ability to strongly activate type I IFN genes, strongly demonstrates that R-DOTAP effectively facilitates antigen cross presentation and also simultaneously activates the type I IFN pathway to drive robust antigen-specific CD8+ T-cell immune responses (FIG. 5).

1-25. (canceled)

26. A method of inducing type I IFN gene expression in a subject comprising administering a composition comprising a cationic lipid and a pharmaceutical carrier to the subject, wherein the cationic lipid activates type I IFN signaling.

27. The method of claim 26, wherein the cationic lipid comprises 1,2-dioleoyl-3-trimethylammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof.

28. The method of claim 27, wherein the cationic lipid is 1,2-dioleoyl-3-trimethylammonium propane (DOTAP).

29. The method of claim 28, wherein the cationic lipid comprises an enantiomer of DOTAP.

30. The method of claim 29, wherein the enantiomer comprises (R)-1,2-dioleoyl-3-trimethylammonium propane (R-DOTAP) or (S)-1,2-dioleoyl-3-trimethylammonium propane (S-DOTAP).

31. The method of claim 26, wherein inducing type I gene expression comprises inducing expression of IFN-α, IFN-β and/or IFN-γ genes.

32. The method of claim 26, wherein a T-cell response is elevated.

33. The method of claim 32, wherein CD8+ T cell responses are elevated.

34. A method for stimulation of a type I IFN signaling pathway in a subject comprising administrating a composition comprising a cationic lipid and a pharmaceutical carrier, wherein the administration of the cationic lipid results in a stimulation of a type I IFN signaling pathway.

35. The method of claim 34, wherein the cationic lipid comprises 1,2-dioleoyl-3-trimethylammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof.

36. The method of claim 35, wherein the cationic lipid comprises (R)-1,2-dioleoyl-3-trimethylammonium propane (R-DOTAP).

37. The method of claim 36, wherein a T-cell response is elevated.

38. The method of claim 37, wherein CD8+ T cell response is elevated.

39. A method for treating cancer in a subject, comprising an administration of a composition comprising a cationic lipid and a pharmaceutical carrier, wherein the administration of the cationic lipid results in a stimulation of a type I IFN signaling pathway.

40. The method of claim 39, wherein the cationic lipid comprises 1,2-dioleoyl-3-trimethylammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof.

41. The method of claim 40, wherein the cationic lipid comprises (R)-1,2-dioleoyl-3-trimethylammonium propane (R-DOTAP).

42. The method of claim 41, wherein stimulation of a type I IFN signaling pathway comprises induction of type I IFN gene expression, wherein the cationic lipid induces the type I IFN gene expression.

43. The method of claim 42, wherein inducing type I gene expression comprises inducing expression of IFN-α, IFN-β and/or IFN-γ genes.
